(19) United States
(12) Patent Application Publication
(10) Pub. No.: US 2021/0095256 A1
(43) Pub. Date: Apr. 1, 2021

(54) METHOD AND SYSTEM FOR 3D CELL CULTURE AND USE THEREOF

(71) Applicant: SSENSTONE INC., Seoul (KR)

(72) Inventor: Dongha Bang, Seoul (KR)

(21) Appl. No.: 16/981,696

(22) PCT Filed: Mar. 19, 2019
(86) PCT No.: PCT/KR2020/003189

§ 371 Date: Sep. 17, 2020

(30) Foreign Application Priority Data
Mar. 22, 2018 (KR) 10-2018-0033594

(51) Int. Cl. C12N 5/09; C12N 5/077; C12N 5/071; G01N 33/50

(52) U.S. Cl. CPC C12N 5/0693; C12N 5/0656; C12N 2513/00; G01N 33/5011; C12N 5/069

(57) ABSTRACT

A system and method for producing 3D organoid using a feeder cell or cell line where the feeder cell or cell line is an endothelial cell, a fibroblast cell or a cell line that is similar to the target cell or the same type of the target cell. The system and method provide rapid culture as well as a long-term sustainable 3D cell or tissue culture environment.

### CROSS-REFERENCE TO RELATED APPLICATIONS

This application claims the benefit under 35 U.S.C. Section 371, of PCT International Application No. PCT/KR2019/003189, filed on Mar. 19, 2019, which claimed priority to Korean Patent Application 10-2018-0033594 filed on Mar. 22, 2018, the disclosures of which are hereby incorporated by reference herein in their entirety.

### BACKGROUND

3D cell culture techniques have led to the creation of more predictive in vitro cell models for numerous applications including cancer research, drug discovery, neuroscience and regenerative medicine. Cells naturally grow and differentiate in three dimensional environments. Cells in their natural environment have constant interactions with extracellular matrix proteins (ECM) and other cells, regulating complex biological functions like cellular migration, apoptosis, or receptor expression. Most of these interactions are lost, or significantly reduced, in traditional 2D cell cultures. Advanced 3D cell systems allow researchers to bridge the gap between classical 2D cell culture and in vivo animal models. Recently, the use of advanced 3D cell culture methods such as tumor spheroids, stem cell organoids and tissue engineering via 3D bioprinting have been implemented to more closely model real in vivo cellular responses. Improving 3D cell culture models to accurately replicate the natural environment will provide more meaningful scientific conclusions and ultimately improve human health. 3D cell culture models can be divided into two main categories: 1) scaffold-based methods using animal derived/synthetic hydrogels or structural 3D scaffolds and 2) scaffold-free approaches using freely floating cell aggregates termed spheroids.

Existing 3D methods are not without limitations, including scalability, reproducibility, sensitivity, and compatibility with high-throughput screening (HTS) instruments. Growing 3D cell models take long time to be utilize as short-term clinical test uses. Also, maintaining 3D cell models in vitro for a long period of time is challenging. Accordingly, a new method and system for 3D cell culture is needed. A 3D cell model which can be cultured in a short period of time can be used for a patient specific medical treatment and can improve efficiency of drug discovery.

The disclosed embodiments of the present disclosure are directed to overcoming one or more of the problems with existing 3D culture methods and systems and illustrate the present invention. The scope of the invention shall not be limited to the disclosed embodiment.

### SUMMARY OF THE DISCLOSURE

One embodiment of the present invention relates to a method for producing 3D cell mass efficiently and effectively using a secondary cell line to support the rapid growth of a target cell to be grown to a three-dimensional structure or three dimensional mass of cells, e.g. organoid, which is herein defined as any 3D mass of cells or 3D cell models including conventional organoid and spheroid as well as tissue. Such 3D cell models have many benefits over conventional two-dimension cell structures. For example, 3D organoid is physiologically more like actual biologic organ or tissue and can be used for drug discovery or screen or medical treatments such as cell therapies, etc. 3D organoid can be used for creating micro physiological systems or organs-on-chips to study difficult clinical problems and development of potential treatments. These human surrogates make use of pumpless self-contained systems that are robust, easy to use, and low cost and allow measurement of metabolic and functional responses.

The second cell or cell line is provided in a medium such as perfused microfluidic medium and placed in a way the second cell or cell line is physically distanced from the target cell for growth. The medium for the target cell and the medium for the second cell may be different but may be the same depending on the target and second cell. The second cell is used to promote the growth of the target cell and is not for own growth. The second cell is an endothelial cell or fibroblast cell, preferably organ specific endothelial cell or fibroblast cell. The endothelial cell is preferably a human dermal microvascular endothelial cell. The endothelial cell or fibroblast cell may be selected from the same or similar organ or tissue from which the target cell is obtained.

The target cell may be placed on a separable substrate plate so that the substrate plate having the target cell can be transferred to a different culture bed. In that way, the cultured target cell can be exposed a new bed with a flash second cell or cell line. The target cell may be any cell from any part of human body such as an organ tissue or skin tissue but may be from a foreign cell such as cancer cell.

The culture of the target cell may be further promoted by providing a third cell in the same culture bed. The third cell may be placed in a medium, which is placed in the culture bed of the target cell. The third cell may be placed in a way that the third cell is distanced from the target cell and the second cell.

The third cell may be selected from the same or similar cell line as the target cell. When the target cell is a cancer cell, the third cell may be a cancer cell line. The third cell may be preferably a single cell line without heterogenecity. The cancer cell line may be a 2D or 3D cell line.

A matrigel mixture may be placed over the medium containing the target cell. The matrigel mixture is a mixture of a matrigel and a fourth medium with a ratio of from about 6:4 to about 8:2.

The culture substrate is a glass bottom dish where a portion of the dish bottom has a glass plate, which can be removed from the glass bottom dish. For example, the portion of the matrigel mixture over the medium containing the target cell may be removed conveniently by lifting the glass plate and relocated into another glass bottom dish to provide a fresh feeder cell. The materials for the glass plate and the glass bottom dish can vary as needed. For example, plastic material may be used.

Another embodiment of the present invention relates to a method for producing 3D organoid by preparing a first medium comprising a target cancer cell; placing the first medium on a growing substrate; placing a matrigel mixture over the first medium; solidifying the matrigel mixture; placing a second medium comprising a cell selected from an endothelial cell, a fibroblast cell or both; placing a third medium comprising a third cell, wherein the third cell is a cancer cell which is similar to the target cancer cell or the same kind as the target cancel cell; placing a conditional medium over the growing substrate to cover the first medium, the second medium and the third medium, resulting in a culture plating; and incubating the culture plating to grow the 3D organoid.

Another embodiment of the present invention relates to a method for producing 3D tissue organoid by providing a first medium comprising a target tissue on a growing substrate and providing a second medium comprising a second cell on the growing substrate. The second medium may be placed in a way that the second medium is physically separated from the first medium to product the 3D tissue organoid.

The second cell in the second medium may be an endothelial cell or a fibroblast cell, preferably an organ specific endothelial cell or fibroblast cell. The endothelial cell may be a human dermal microvascular endothelial cell.

The method may include a third medium comprising a third cell comprising a third cell and the third medium may be placed physically away from the first medium and the second medium.

The third cell is preferably a cell similar to the target cell or the same type cell as the target cell where the conditions for the third cell are adjusted for the third cell's growth. The third cell may be a cell line containing more than one type of cell.

The first target cell may be a cancer cell and the third cell may be a cancer cell line. Preferably, the cancer cell line is a single cell line without heterogenecity. The cancer cell line is a 2D or 3D cell line.

The method may include providing a matrigel mixture over the first medium wherein the matrigel mixture is a mixture of a matrigel and a fourth medium with a ratio of from about 6:4 to about 8:2.

Another embodiment of the present invention is a system for growing 3D organoid including a first medium comprising a target cell or tissue and a second medium comprising a second cell. The first medium and the second medium may not be directly in contact each other. The second cell may be selected from an endothelial cell, a fibroblast cell, a cell line that is similar to the target cell or the same type of the target cell, and the system is used to grow the 3D organoid.

The system may include a third medium comprising a third cell. The third medium may not be directly in contact with the first medium or the second medium. The third cell is selected from an endothelial cell, a fibroblast cell, a cell line that is similar to the target cell or the same type of the target cell and different from the second cell.

The system may further include a matrigel mixture, which is a mixture of a matrigel and a fourth medium with a ratio of from about 6:4 to about 8:2.

Another embodiment of the present invention is a microphysiology system, organ-on-chip device. The device or system is based on physiologically based on pharmacokinetic models and use human cell or tissue engineered constructs placed in a microfabricated device. The device is self-contained systems that are robust, easy to use, and low cost and allow measurement of metabolic and functional response. The device may be used for drug screen or further medical treatment development.

Another embodiment of the present invention relates to a method for screening a proper cancer drug using a 3D cancer organoid by a) providing a 3D cancer organoid cultured by using a culture system wherein the system comprises of a first medium with a target cell or tissue and a second medium with a second cell, wherein the first medium and the second medium are not directly in contact each other, and wherein the second cell is selected from a endothelial cell, a fibroblast cell, or a cell line that is similar to the target cell or the same type of the target cell b) applying a medical substance to the 3D cancer organoid; and c) determining a pharmaceutical effect of the medical substance on the 3D cancer organoid.

### BRIEF DESCRIPTION OF THE DRAWINGS

FIG. 1 is an illustration of an embodiment according to the present disclosure.

FIG. 2. (a) is a side view of an illustration of an embodiment according to the present disclosure and (b) is another side view of an embodiment with a removable plate.

FIG. 3 is an illustrative flow chart utilizing an embodiment according to the present disclosure.

FIG. 4 is an illustrative flow chart utilizing an embodiment according to the present disclosure.

FIG. 5 is an illustrative flow chart utilizing an embodiment according to the present disclosure.

FIG. 6 shows pictures of human lung cancer cell colonies grown in 3D according to an embodiment of the present disclosure.

FIG. 7 is a photography of a culture for 3D human lung cancer cell colonies.

FIG. 8 shows pictures of mouse lung stem cell colonies grown in 3D. (a) shows the colonies after 7 days and (b) shows after 14 days.

FIG. 9 shows 3D mouse lung whole tissue culture: (a) shows at day 0; (b) shows at day 7; (c) and (d) shows vessel growth.

FIG. 10 shows pictures of 3D mouse lung whole tissue culture after 50 days.

FIG. 11 shows pictures of mouse liver duct stem cell colonies grown in 3D culture.

FIG. 12 shows pictures of mouse spermatogonial stem cell colonies grown in 3D culture.

FIG. 13 shows pictures of mouse pancreatic ductal stem cell colonies grown in 3D culture.

FIG. 14 illustrates multi-culture wells for a drug screen.

FIG. 15 shows (a) a culture result without a medicinal treatment and (b) a culture result with a medical treatment for the same period as (a).

### DETAILED DESCRIPTION OF PREFERRED EMBODIMENTS

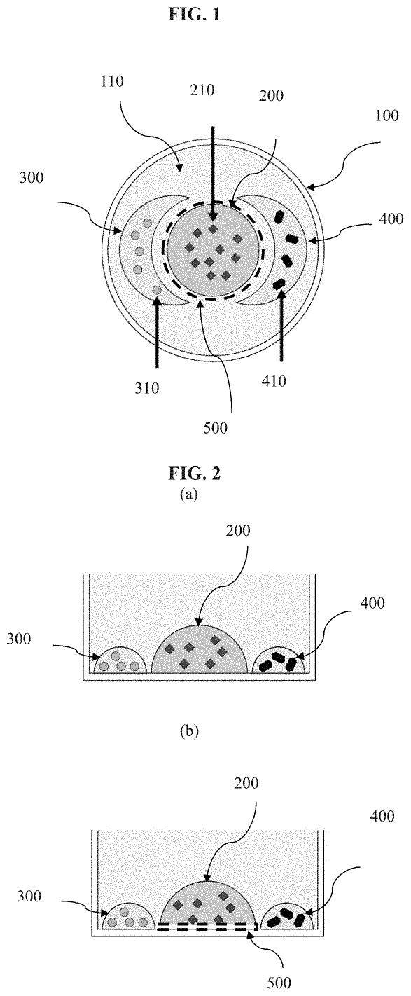

FIG. 1 illustrates a culture system according to one embodiment of the present invention. Target cells, such as a cancer cell from a patient, are obtained from a patient and separated into a small piece. The separated cells 210 are placed in a conditioned medium such as a microfluidic medium and in matrigel. The mixture 200 is placed in a glass bottom dish 100 and incubated to solidify the mixture on the dish. A feeder cell or cell line 310 in a conditioned medium and a matrigel is provided in the same dish but can be placed in a way that the feeder cell or cell line mixture 300 is separated from the target cell mixture 200. The feeder cell or cell line is not necessary from the patient but from a third party. The separation allows harvesting cultured target cells without any contamination of cells from the feeder mixture.

The feeder cell or cell line is to promote the growth of the target cell and is not for own growth. The feeder cell or cell line is preferably an endothelial cell or fibroblast cell. More preferably, the endothelial cell is a human dermal microvascular endothelial cell. Even more preferably, the feeder cell or cell line is an endothelial cell or fibroblast cell from an organ that is similar to or the same as the organ from which the target cell is obtained.

The culture bed may have a second feeder cell or cell line 410 which is similarly prepared in a conditioned medium and matrigel. The second feeder cell or cell line mixture 400 may also be placed in a way that the mixture 400 is separated from the target cell or cell line mixture. The second feeder cell or cell line can be placed in the dish. The second feeder cell or cell line is preferably cell or cell lines that is similar to or the same cell or cell line as the target cell. For example, a publicly available cancer cell line with known conditions for the conditioned medium may be used for a 3D culture of a cancer cell. Since the target cell mixture bed is separated from the feeder cell beds, cultured target cells can be harvested with any contamination from the feeder cell beds.

Optionally, the mixture 200 is placed on a separate removable plate 500, which allows removing the mixture bed from the dish and relocate to another dish to provide additional feeder cells or nutrients.

After the target cell and feeder cell mixtures are placed in the dish, a conditioned medium can be filled in the dish. Or a mixture of a matrigel and another medium with a ratio of from about 6:4 to about 8:2 may be filled in the dish.

Media used in herein may need to be adjusted depending on cell or cell line types. There are many ingredients that can go into the media. For example, some of the following components in Table 1 may be combined to form a proper media:

TABLE 1
Typical Ingredients for 3D Culture Media

StemPro ™-34 (www.thermofisher.com)
Growth factors
StemPro ™ 34 + nutrients + GPS
1% Fetal Bovin Serum (FBS) (heat inactivation)
5 mg/ml Bovine Serum Albumin (BSA)
Insulin 25 mg,
Transferrin 25 mg,
25 ug Selenious acid
Noggin: 100 ng/ml
N-Acethylcystein 1.25 mM
Advanced DMEM (Dulbecco Modified Eagle Medium)
(Thermo Fisher Scientific)
ECGS (Biomedical Technologies, Stoughton MA)
25 ml HEPES (Sigma),
100 ug/ml heparin (Sigma)
DMEM/F12 (Thermo Fisher Scientific)
Penicillin(100 IU/ml)-streptomycin(100 ug/ml)
2% NuSerum ™ (BD Biosciences)
ACL-4 media (Thermo Fisher Scientific)
RPMI 1640 (Sigma)
EGM2 media (Lonza Bioscience)
EBM media (Lonza Bioscience)

There are many growth factors that can be utilized in the media. For example, one or more of the following growth factors can be used: Human EGF 10-100 ng/ml, Human FGF10 10-100 ng/ml, Human FGF2 10-100 ng/ml, Human FGF110-100 ng/ml, Human HGF 10-100 ng/ml, Human VEGF 10-100 ng/ml, GDNF 20 ng/ml, SDF-1 (CXCL12) 50 ng/ml, CXCL1 10 ng/ml, CXCL2 10 ng/ml, NGF, PDGF, IGF-1, and TGF-beta.

The conditioned medium can be chosen from various media. Many cell culture medium formulations are documented in the literature and a number of media are commercially available. Once the culture medium is incubated with cells, it is known to those skilled in the art as “spent” or “conditioned medium”. Conditioned medium contains many of the original components of the medium, as well as a variety of cellular metabolites and secreted proteins, including, for example, biologically active growth factors, inflammatory mediators and other extracellular proteins.

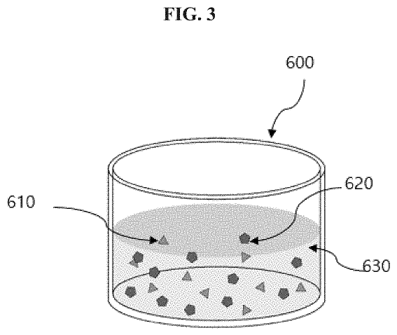

When the cultured target cells do not require to be separated from foreign cells from the feeder cells, the feeder cell or cell line mixtures 610 may be mixed with the target cell mixture 620 in a conditioned media 630 as the illustrated culture bed 600 in FIG. 3.

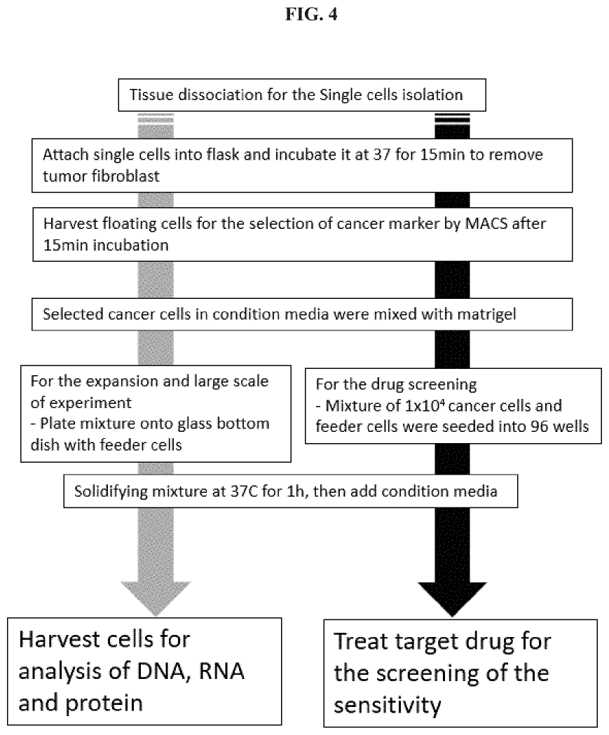

FIG. 4 illustrates example protocols for 3D culture and uses of cultured cells. Tissue from a patent is dissociated for a single cell isolation. The single cells are seeded into a flask and the attached flask is incubated at 37° C. for 15 minutes to eliminate tumor fibroblast. After this step, floating cells are harvested for cancer marker selection through MACS® technology (https://www.miltenyibiotec.com/DK-en/products/macs-cell-separation.html. Selected cancer cells in a conditioned medium are mixed with matrigel. For an expansion and large scale of experiment, the matrigel mixture is plated on a glass bottom dish with feeder cells as described above. For a drug screening, a matrigel mixture with 1×10⁴ cancer cells and feeder cells is plated into 96 wells. Then, the mixture may be solidified at 37° C. for 1 hr., and a conditioned medium is added on the top of the solidified mixture.

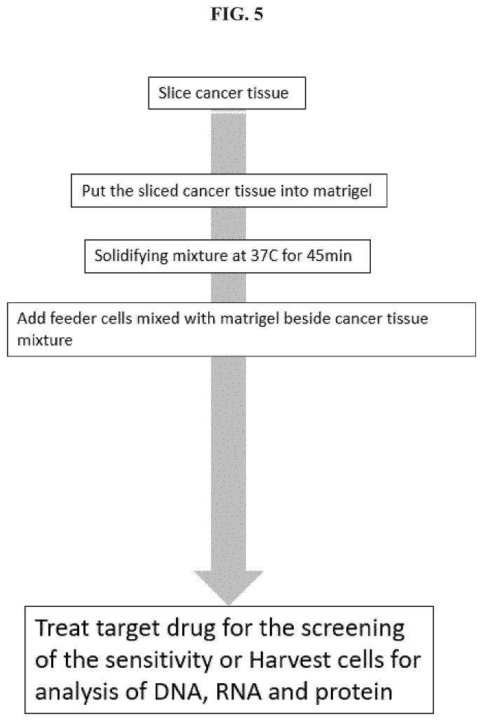

The 3D culture method and system of the present invention can be used to culture tissues. For example, FIG. 5 illustrates a method for 3D culture of tissue. A tissue sample is obtained by slicing cancer tissue from a patient. The tissue sample may be placed in matrigel, of the mixture may be solidified at 37° C. for 45 minutes. Then, feeder cells mixed with matrigel are placed as described herein for 3D culture. The cultured tissue can be used for drug screening or sensitivity tests or cell harvesting for other tests or uses

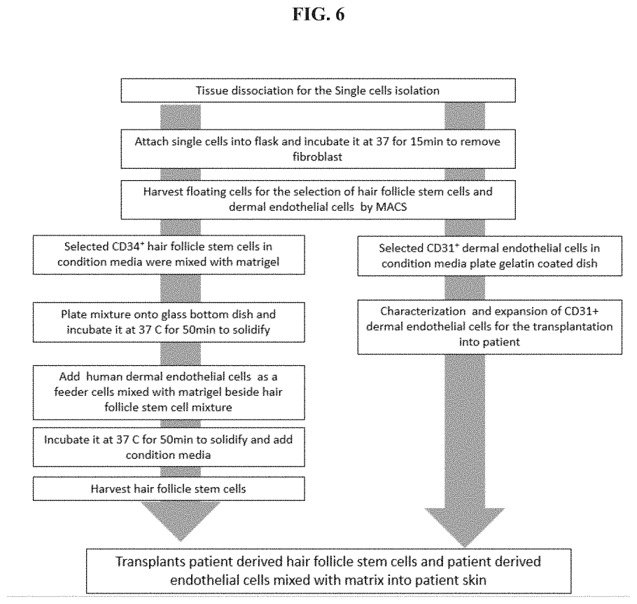

An embodiment of the present invention can be used to culture cells or tissues for direct transplantation to a patient. For example, FIG. 6 illustrates an example protocol for culturing hair follicle stem cells and dermal endothelial cells and transplanting them to a patient's skin. In this case, the target cell and the feeder cell can be obtained from a patent to whom cultured cells are to be transplanted. A hair tissue sample from a patient is obtained and the hair tissue sample is subject to the single cell isolation process and using cell separation technique such as MACS, hair follicle stem cells and dermal endothelial cells are respectively harvested. The dermal endothelial cells are cultured to increase cell mass, which can be used as a feeder for the hair follicle stem cell culture as well as to be transplanted to the patient along with the cultured hair follicle stem cells to regrow hair in the patient.

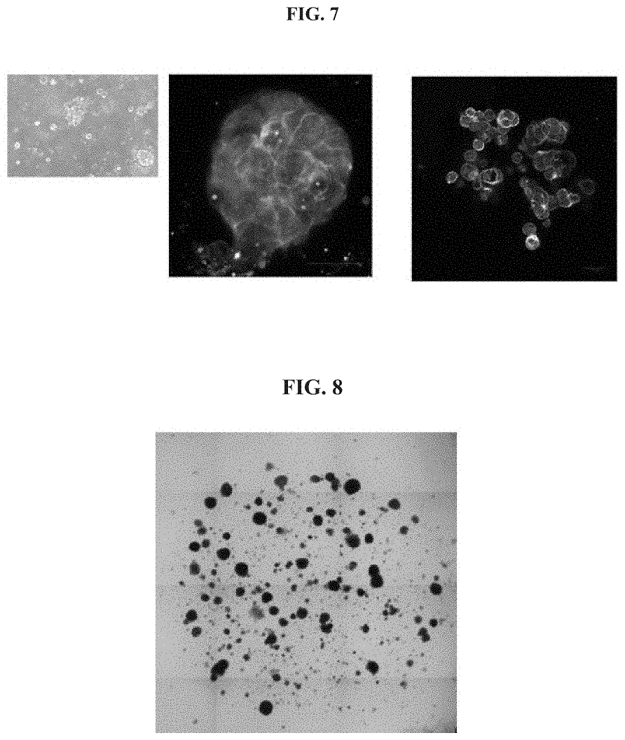

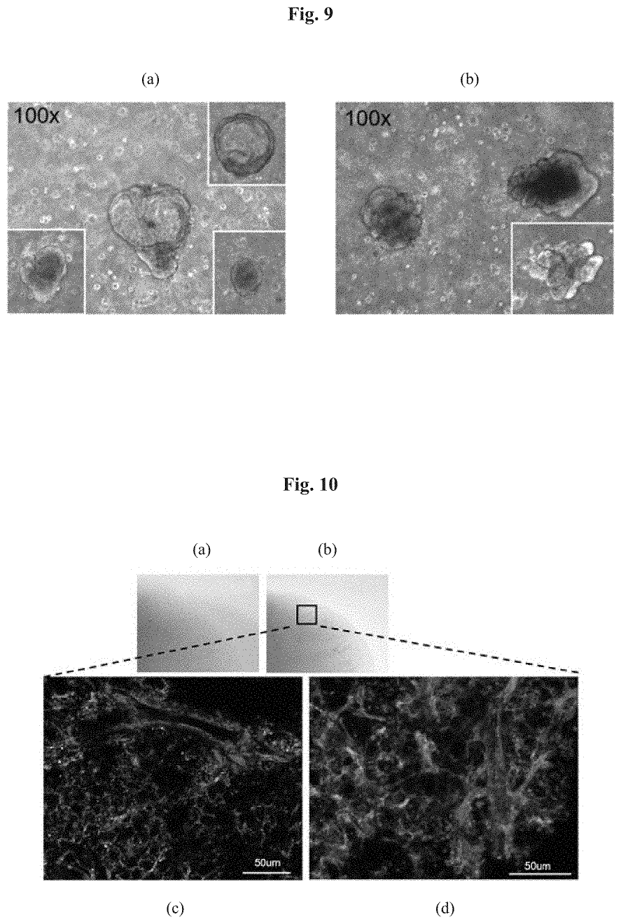

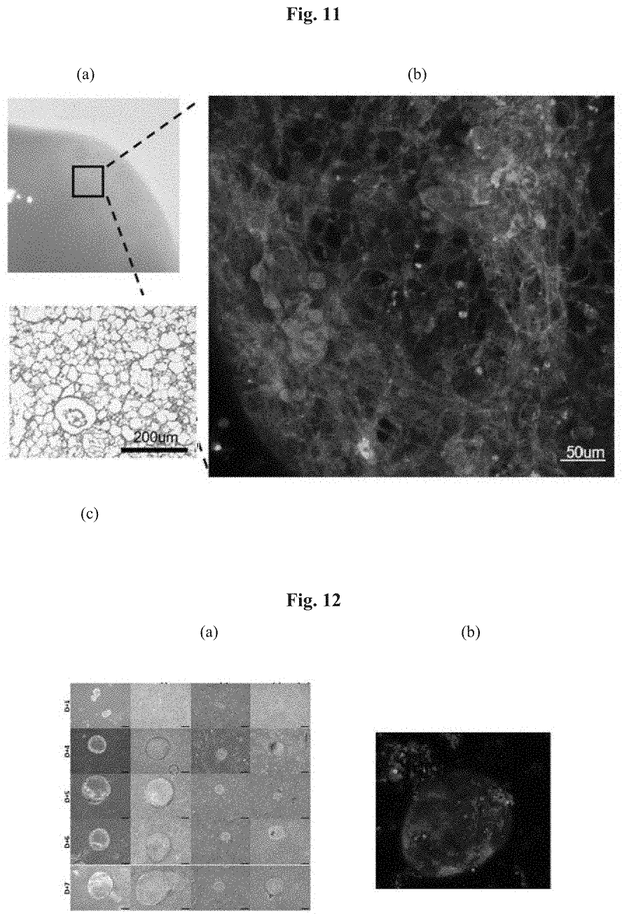

Human lung cancer cells have been cultured using an embodiment of the present invention. FIGS. 7 and 8 show the human lung cancer call colonies grown according to an embodiment of the present invention. Similarly, FIGS. 9, 10, 11 show mouse lung stem cell colonies and whole issue grown by utilizing an embodiment of the present invention. FIG. 12 (a) shows different sizes of 3D cultured mouse liver ducts from day 2 to day 7. FIG. 12 (b) is an immunofluorescence image of a 3D cultured mouse liver duct.

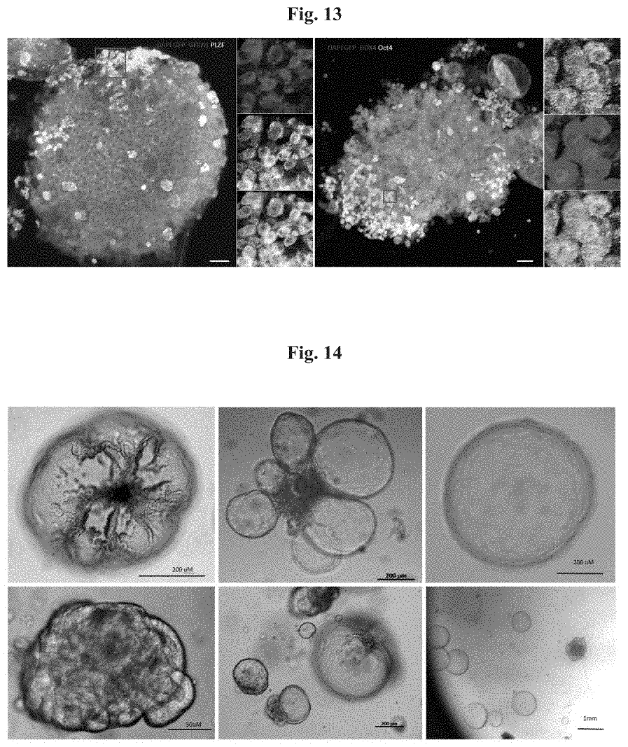

FIGS. 13 and 14 are pictures of mouse spermatogonial stem cell colonies and mouse pancreatic ductal stem cell colonies respectively grown in 3D culture.

The various stem cell colonies grown in 3 D culture can be used in stem cell treatment, drug discovery, drug sensitivity test, and other stem cell science. In particularly, the 3D culture methods and systems according to the present invention allow grow stem cell colonies or tissue in a shorter period of time than the conventional methods and systems. The speed is particularly important to utilize the method and system as patient-specific medicine because, for example, a cancer patient would require a quick drug screen or cell therapy as cancers usually grow very rapidly.

Also, the 3D culture systems and methods according to the present invention sustain the cultured cells or tissue for a long period of time. If necessary, the cultured cells o tissues can live several months, even longer. This is a very important feature for drug discovery or tests.

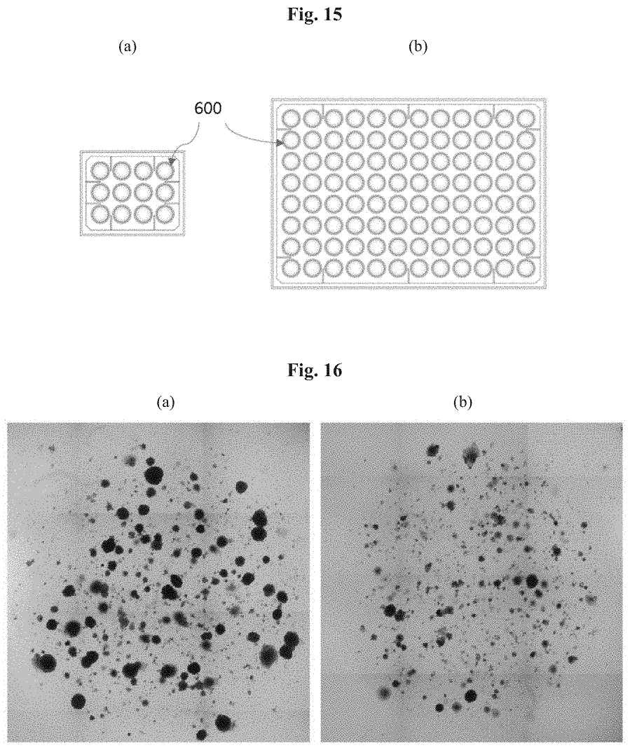

For drug discovery or test, multi culture wells can be used. FIG. 15 shows two different multi culture wells examples. Each well 600 can have a 3D culture system. As each well can have the same 3D culture system, these systems are often used to compare reactions to different substance, e.g., cancer drug. FIG. 16 shows that cancer cells grow differently with and without a medical treatment where the cells in (b) show lack of growth comparing to those in (a).

The following examples are illustrative purpose only. The scope of the invention should not be limited to these examples.

### Example 1. Lung Cancer Cell Isolation

a. Mincing tumor tissues with scissor as small as possible and put it into dissociation solution.
   Dissociation solution: collagenase type II/DNasel in HBSS
b. Incubate minced tumor tissue at 37 C for 40 min and add same volume of FBS for the neutralization.
c. Filtered digested tissue through 100 uM.
d. Spin filtered single cells with 1000 rpm for 10 min.
e. Wash cell pellet with PBS and spin down at 1000 rpm for 5 min (3 times).
   i. If you can find RBC in the pellet, use RBC lysis buffer as following commercial instruction.

### Example 2. Plating Lung Cancer Cells with Feeder Cells (HMVEC and Lung Cancer Cell Line)

a. Count number of single lung cancer cells from cancer tissue
b. Prepare 1×10⁵ of HMVECs for feeder cells
c. Put 100 ul of HMVECs (5×10⁵) in matrigel mixture including HMVEC condition media (20% of total mixture) inside of dish and incubate it at 37 C for 45 min
d. 1×10⁵ of Single lung cancer cells were mixed with matrigel and conditioned media containing EGF/FGF-2 growth factors.
e. Put the mixture of lung cancer cells on center of 96 well or 12 well and incubate it at 37 C for 45 min.
f. Add conditioned media into well after solidifying 3D lung cancer cells mixture.
g. Add drug as a following concentration into 96 well and 12 well
   ii. Treat drug into 3d organoid every 3 days for 5-7 days.

What is claimed is:

1. Method for producing 3D organoid, comprising
a. providing a first medium comprising a target cell or tissue on a growing substrate and
b. providing a second medium comprising a second cell on the growing substrate, wherein the second medium is placed in a way that the second medium is physically separated from the first medium to product the 3D organoid.

2. The method according to claim 1, wherein the second cell in the second medium is an endothelial cell or a fibroblast cell.

3. The method according to claim 1, wherein the second cell is an organ specific endothelial cell or fibroblast cell.

4. The method according to claim 1, further comprising a third medium comprising a third cell comprising a third cell wherein the third medium is placed physically away from the first medium and the second medium.

5. The method according to claim 4, wherein the third cell is a cell similar to the target cell or the same type cell as the target cell.

6. The method according to claim 1, where the target cell is a cancer cell.

7. The method according to claim 6, wherein the second cell in the second medium is an endothelial cell or a fibroblast cell.

8. The method according to claim 7, further comprising a third medium comprising a third cell comprising a third cell wherein the third medium is placed physically away from the first medium and the second medium wherein the third cell is a cancer cell line.

9. The method according to claim 8, wherein the endothelial cell is a human dermal microvascular endothelial cell.

10. The method according to claim 8, wherein the cancer cell line is a single cell line without heterogenecity.

11. The method according to claim 10, wherein the cancer cell line is a 2D or 3D cell line.

12. The method according to claim 11, further comprising providing a matrigel mixture over the first medium wherein the matrigel mixture is a mixture of a matrigel and a fourth medium with a ratio of from about 6:4 to about 8:2.

13. The method according to claim 1, wherein a removable plate is placed on the grow substrate.

14. A method for producing 3D organoid, comprising
a) preparing a first medium comprising a target cancer cell;
b) preparing a second medium comprising a cell selected from an endothelial cell, a fibroblast cell or both; and
f) placing the first medium and the second medium in a grow substrate;
g) placing a conditioned medium over the growing substrate to cover the first medium and the second medium, resulting in a culture plating; and
f) incubating the culture plating to grow the 3D organoid.

15. A system for growing 3D organoid, comprising
a) a first medium comprising a target cell or tissue and
b) a second medium comprising a second cell
wherein the first medium and the second medium are not directly in contact each other,
wherein the second cell is selected from an endothelial cell, a fibroblast cell, a cell line that is similar to the target cell or the same type of the target cell, and
wherein the system is used to grow the 3D organoid.

16. The system according to claim 15, further comprising a third medium comprising a third cell wherein the third medium is not directly in contact with the first medium or the second medium and the third cell is selected from a endothelial cell, a fibroblast cell, or a cell line that is similar to the target cell or the same type of the target cell and different from the second cell.

17. The system according to claim 16, further comprising a matrigel mixture, which is a mixture of a matrigel and a fourth medium with a ratio of from about 6:4 to about 8:2.

18. A method for screening a proper cancer drug using a 3D cancer organoid, comprising
a) culturing a target cancer cell to 3D organoid using a culture system wherein the system comprises of a first medium with a target cell or tissue and a second medium with a second cell, wherein the first medium and the second medium are not directly in contact each other, and wherein the second cell is selected from an endothelial cell, a fibroblast cell, a cell line that is similar to the target cell or the same type of the target cell
b) applying a medicine to the 3D cancer organoid; and
c) determining its effect on the 3D cancer organoid.
